(19) United States
(12) Patent Application Publication
(10) Pub. No.: US 2007/0160999 A1
(43) Pub. Date: Jul. 12, 2007

(54) Method for simultaneous extraction of nucleic acids from a biological sample

(76) Inventor: Fiorella Calabrese, Selvazzano Dentro (IT)

Correspondence Address: BRYAN W. BOCKHOP, ESQ., 2375 MOSSY BRANCH DR., SNELLVILLE, GA, 30078, US

(21) Appl. No.: 10/588,120

(22) PCT Filed: Feb. 2, 2005
(86) PCT No.: PCT/IT05/00048

§ 371 Date: Aug. 1, 2006

(30) Foreign Application Priority Data
Feb. 4, 2004 (IT) MI2004A000167

(51) Int. Cl. C12Q 1/68; C12N 1/08

(52) U.S. Cl. 435/6; 435/270

(57) ABSTRACT

A new method of simultaneous and separate extraction of deoxyribonucleic acid (DNA) and ribonucleic acid (RNA) from a biological sample, indifferently from fresh, frozen, fixed or autoptic tissue with a weight no less than 5 mg. The main steps of the method are: 1/sample lysis in a solution composed of a caotropic agent (urea or guanidine salt), a ionic detergent (SDS or SLS), a proteolytic enzyme (proteinase K, trypsin, chymotrypsin, pepsin or pronase), a reducing agent (β-mercaptoethanol or dithiothreitol); 2/deproteination; 3/precipitation of RNA from aqueous phase; 4/precipitation of DNA from organic phase. The invention includes an extraction kit for nucleic acids, also of viral origin.

### TECHNICAL FIELD

The invention relates to a biochemical method for the simultaneous and separate extraction of nucleic acids (deoxyribonucleic acid and ribonucleic acid) from a biological sample.

### BACKGROUND ART

Modern molecular biology has been revolutionary in traditional biology and many fields of biomedical sciences. In recent years the union between medical biosciences and clinical practice has become even more practical and immediate. The recent application of recombinant DNA techniques is widespread in many different diagnostic fields, allowing the achievement of quicker and more accurate diagnoses. Some of the most common diagnoses provided by molecular biology laboratories are: the identification and quantification of many viral agents (both DNA and RNA viruses), the demonstration of oncogene over-expression (considered to be of prognostic value in many neoplasias), the precise characterization of genetic diseases (by detection of gene mutations and deletions) and the assessment of monoclonality in lymphomas.

The available techniques allow DNA/RNA extraction from cells/tissue of different origins. Different protocols can be used, commercialised as “kits”, which can be used to extract nucleic acids from different material (biological fluids, cell cultures, fresh or frozen tissue samples). The quality and quantity of nucleic acids extracted are the pivotal point for performing “non in situ” molecular techniques such as Polymerase Chain Reaction (PCR). Biopsy samples usually have a low weight (often less than 5 mg) which does not guarantee that the quantity of nucleic acids will be sufficient for diagnostic purposes. Most of the tissue and cytological samples are formalin-fixed and paraffin-embedded and they constitute a pathological tissue archive. During fixation, nucleic acids are heavily degraded making it difficult to perform subsequent molecular reactions. Inasmuch, a bad quality of nucleic acids may compromise the molecular reactions (Volenandt et al., “Polymerase chain reaction of DNA from paraffin-embedded tissue”, Methods in molecular biology vol. 15: Current methods and application, 1993, edited by B A White, Humana Press Inc, NJ). RNA extraction is usually more difficult due to the ubiquity of RNases and its intrinsic fragility at alkaline pH. Viral RNA is even more fragile and quantitatively inferior (smaller number of copies) to the native one in infected cells (Mizuno T et al., “RNA from decades-old archival tissue blocks for retrospectives studies”, Diagn Mol Pathol 1998; 7:202-208). Another parameter which can heavily influence the yield of the nucleic acid extraction yield is the fixation time (Foss R D et al., “Effects of fixative and fixation time on the extraction and polymerase chain reaction amplification of RNA from paraffin-embedded tissues”, Diagn Mol Pathol 1998; 7:184-188); a prolonged lysis protocol may allow a larger quantity of nucleic acids to be obtained. Some commercial kits with a very short extraction time, a short lysis period and no deproteinization steps, may invalidate the extraction procedure, especially if performed on archival tissue. Moreover, these procedures can themselves be a cause of impurities which can interfere with the application of other molecular biology techniques. The simultaneous extraction methods, both for DNA and RNA, which are used selectively on fresh or frozen tissues, cells or biological fluids, have some drawbacks. The method reported by Coombs L M et al. (“Simultaneous isolation of DNA, RNA and antigenic protein exhibiting Idnase activity from small tumour samples using guanidine isothiocyanate”, Anal. Biochem 1990; 188:338-343) is based on ultracentrifugation of a homogeneous sample and a caesium guanidine-chloride solution. Only a small number of samples can be prepared with this protocol. Another simultaneous method of both DNA and RNA extraction from the same tissue sample has been reported by Chornzynski (U.S. Pat. No. 5,346,994). With this method the tissue is homogenized in a phenol and guanidine-isothiocyanate solution, followed by the addition of chloroform and with the subsequent separation by ethanol of DNA (interphase), RNA (water phase) and proteins from the organic phase. The same inventor in WO 97/05248 proposes a method without phenol employment and based only on the use of a caotropic agent such as guanidine-isothiocyanate, reducing agents such as 2-amino-ethanthiol (replaceable with mercapto-ethanol) and a buffer such as Na-acetate, sarcosil 0.2% and isopropanol. Nucleic acid precipitation is accomplished with isopropanol and the RNA pellet is stored in formamide at −20° C., whereas the DNA is solubilized with NaOH and neutralized with HEPES. This method assures a 91% DNA recovery.

In WO 91/02740 the author proposed a lysis solution containing 4M guanidine-isothiocyanate, 0.1M mercapto-ethanol, 25 mM Na-citrate, 0.5% sarcosine, 0.5M Na acetate and a polyanion for deproteination. In the protocol, the use of 100 μgr/ml of proteinase K is suggested. DNA precipitation is carried out by standard methods (ethanol or isopropanol) whereas for RNA precipitation ethanol and the DEPC water are used. No purifications with phenol/chloroform are performed and a different precipitation of nucleic acids is obtained, particularly DNA which seems to be preferentially extracted. The simultaneous extraction of a sufficient quantity of both DNA and RNA from the same biological sample is particularly important to answer diagnostic questions (i.e. myocarditis by DNA or RNA viral agents, mutation or over-expression of a given oncogene). Only a few protocols or commercial kits actually available can perform the simultaneous extraction of DNA and RNA and their utility is limited by the low extraction yield and the narrow field of application: fresh biological fluids, cell cultures and fresh/frozen tissue. No protocols of simultaneous extraction of both DNA and RNA from formalin-fixed paraffin-embedded tissues are actually available. This type of tissue is the main source for routine pathological exams and retrospective studies of diagnostic and research utility. For all these reasons it is becoming particularly urgent to realize the simultaneous extraction of both DNA and RNA from any tissue sample, even if formalin-fixed and paraffin-embedded. The extraction efficiency must be very high: the new method proposed in the invention has a very high extraction yield and allows the extraction of large amounts of nucleic acids (both DNA and RNA) from extremely small samples of fresh/frozen tissue.

### DISCLOSURE OF INVENTION

The invention relates to a method of simultaneous and separate extraction of deoxyribonucleic acid (DNA) and ribonucleic acid (RNA) from a biological sample (biopsy, fragment or section), indifferently for fresh, frozen, fixed or autoptic tissue with a weight not less than 5 mg. Surprisingly, this method provides a good extraction yield (both quantitatively and qualitatively) of both nucleic acids, even from autoptic tissue.

According to the method, the biological material should be minced (but not homogenized), denaturated by incubation in a lysis containing: a caotropic agent (urea or guanidine salt), an ionic detergent (SDS or SLS), a proteolytic enzyme (proteinase K, trypsin, chymotrypsin, pepsin, pronase), preferably proteinase K, and a reducing agent. The preferred ionic detergent is SLS, with a concentration of 0.01-2%, more preferably 0.2-1%. The preferred caotropic agent is guanidine salt and even better is guanidine thiocyanate with a concentration of 1-4 M; the proteolytic enzyme is preferentially proteinase K in a concentration of 0.1-10 mg/ml and even better 0.5-0.8 mg/ml. In particular, proteinase K is added up to a final concentration of 1-2 mg/ml when the tissue is fresh or frozen and up to 4-6 mg/ml when the tissue is fixed. The lysis solution contains a reducing agent such as DTT or β-mercapto-ethanol, preferably the latter.

The preferred buffer used in the lysis solution is NA-citrate with a concentration of 5-100 mM, preferably 10-35 mM. The pH is neutral, between 6.8 and 7.3, more preferably between 6.9 and 7.17.

According to step 1 of the procedure the sample is added to the lysis solution, which also contains ribonuclease inhibitors, such as Vanadyl-Ribonucleoside Complex, with a known final concentration between 10-200 μM and preferably between 50 and 100 μM, and an agent useful in precipitating nucleic acids, like tRNA or glycogen, preferably the latter, in a concentration of 1-200 ng/ml and even better if 50-100 ng/ml. The addition of this precipitating agent can be done in a subsequent passage, before nucleic acid precipitation using alcohol.

The temperature of the solution should be maintained over 15° C., preferably higher than 25° C., and even better if higher than 30° C. and comprised between 35 and 42° C. for at least 5 hours and better if more than 10. When tissue is particularly resistant to lysis, especially if it is fixed, the incubation time can be prolonged and optionally an aliquot of the proteolytic enzyme can be added. If the tissue or the biopsy are paraffin-embedded, the sample must be deparaffinated before adding the sample to the lysis solution. The sample is cut with a microtome into a certain number of sections weighing between 0.5 and 2 mg, corresponding to 15-30 10 μm sections for biopsies and to 1-4 10 μm sections for fragments. The sections are deparaffinated with xylol or with commercially available reagents, like Histoclear™, or benzene-derived substances, at a temperature of 30° C., preferably if higher than 35° C. After deparaffination with xylol, the sample is washed with the same volume of alcohol, preferably absolute ethyl alcohol, or acetone.

After deparaffination, the sample can be treated according to step 1 of the procedure as if it was a fresh or frozen sample, and it can be added to the lysis solution.

If the sample is formalin-fixed but not paraffin-embedded, it can be treated with alumina (Al₂O₃). The sample is removed from the fixative solution (i.e. formalin) and it is dried in an oven for 2 hours at 30-35° C. and then treated with alumina (added in a weight equal to the sample's). The compound is mixed for few minutes and the mixture is incubated in the lysis solution and processed according to the new method. The alumina powder abrades the tissue during mixing and favours the tissue and cellular fragmentation leading to cell membrane rupture and the spilling out of intracellular material thus comprising the nucleic acids. The new method was successfully adopted to extract nucleic acids, particularly viral, from heart tissue fragments formalin-fixed for one year (FIG. 5). According to the main steps of the procedure, after denaturation in the lysis solution, the sample is deproteinated according to step 2 of the procedure, by adding the same volume of phenol or a mixture of phenol-chloroform (with a volume/volume ratio between 3:1 to 7:1, preferably 5:1) to the solution, keeping an acid pH, preferably under 5, mixing repetitively the water and the organic phases, according to the literature. The water phase ca optionally be re-extracted by admixing chloroform again to eliminate phenol remnants.

The RNA is then precipitated from the water phase/phases according to step 3 of the procedure, by adding short chain aliphatic alcohols, such as propylic acid or ethanol (preferably isopropanol) keeping the tube at −20° C. preferably less than −50° C. and better if −80° C. Optionally the phenol and phenol-chloroform extraction can be repeated again. The organic phases containing phenol are kept for DNA extraction which is subsequently performed according to step 3 of the procedure. DNA precipitation is carried out by adding absolute ethanol and a precipitating agent, like tRNA or glycogen (at the above indicated concentration), to the organic phase and then incubating for a few minutes at room temperature.

Alcohol volumes added for nucleic acid precipitation are chosen according to proportions usually known by the technicians operating in a molecular biology laboratory. Other quantities, concentrations or solutions not explicitly specified in the presented method can be easily determined by a specialised technician, according to what is published in many molecular biology technique handbooks (i.e. Sambrook and Maniatis, Molecular Cloning, 1988, CSH Edition).

Surprisingly, the new method allows the simultaneous extraction of a quantity of nucleic acids superior to those achievable with other protocols or commercial kits. Particularly, the nucleic acids obtained are of good quality, even if they are extracted from unsuitably preserved samples, i.e. sample formalin-fixed for more than 3 days up to a few years. The extraction yields of the new method are superior to the commonest simultaneous extraction methods, directly compared in the experimental phase of the present method or on the basis of given yields. The new method is superior also if compared to methods optimised for the extraction of a single nucleic acid (DNA or RNA), such as the Blin and Stafford and Chomczynsky and Sacchi's methods, whose yields are shown in Table 5. Simultaneous allows the extraction of a sufficient quantity of nucleic acids even if the starting material is a low in weight. Other advantages of the new method are the low concentrations of the denaturating agent in the lysis solution and the absence of ultracentrifugation, resulting in easy applicability of the method even in less equipped laboratories. The method of invention is also useful when nucleic acids must be extracted form autoptic samples because the extractive yield is always good both in quantity and quality. With the method, viral nucleic acids, including RNA, usually present in a number of copies largely inferior to endogenous mRNA, could be extracted from autoptic samples. The extraction yields obtained with the new method, compared with other known methods, have been evaluated for fragments with a weight between 0.5 and 20 mg extracted with a quantity of lysis solution not inferior to 300μl.

The total quantity of RNA extracted with the invention method was at least of 15 μg. Starting from a 10 mg sample, the RNA quantity was at least 20 μg and sometimes reached even 50 μg (53.8 μg). The extraction yields of DNA, calculated from the minimal useful quantity of tissue (10 mg), were at least 1 μg and sometimes up to 10 μg.

For fixed material the yields varied from 2.5 to 25 μg of RNA for tissue sections not inferior to 20 mm² and between 8.8 and 26 μg for tissue sections larger than 20 mm² (up to 1 cm²). The DNA extraction yields varied from 0.2 to 1.6 μg for tissue sections not inferior to 20 mm² and between 1.6 and 8.2 μg for tissue sections larger than 20 mm.

The quality of nucleic acids extracted with the new method has been verified by PCR with specific oligonucleotides for house keeping genes (genes which are always expressed in all tissues) and through an analytical method which evaluates the ratio A260/A280, usually ranging from 1.4 to 2, (preferably from 1.5 to 1.8). During PCR reaction the RNA has been previously retrotranscripted by an inverse retrotranscriptase. The nucleic acids obtained with the new method are adequately purified and can be used for further molecular biology application. This can be demonstrated by the positive PCR reaction of nucleic acids obtained (100% of fresh/frozen samples, 93% of fixed samples) and by an optimal A260/A280 ratio in most of the samples.

The new procedure can be briefly described as follows: after the sample has been minced (but not homogenized) with a lancet sterile blade.
a) it is incubated in lysis solution as described above. This admixture of the sample in the lysis solution can also be defined as the aqueous phase if there is the addition or contact with an “organic phase” (phenol, phenol-chloroform, chloroform). Optionally, if macroscopically the sample is not lysed an aliquot of proteolytic enzyme can be added and then the incubation is repeated.
b) the aqueous phase is extracted with a deproteination admixture made of phenol or phenol-chloroform at acid pH (organic phase). Optionally, the water can be re-extracted with chloroform, repeating this passage at least twice. Optionally some water solutions or water treated with RNases inhibitors (i.e. DEPC, DiEthylPyroCarbonate) can be added to the organic phase. Then the organic phase/phases can be stored for DNA extraction.
c) the aqueous solution and/or H₂O as previously described is added to the lysis solution containing the sample and the ribonucleic acid (RNA) is precipitated by the addition of a short chain aliphatic alcohol (preferably isopropanol) and a precipitating agent (as tRNA or glycogen), at the final concentrations described above. The exceeding salts can be removed from the precipitate by repeated washings with short chains aliphatic alcohols water-diluted, preferably water-diluted ethanol at 70-80%. The addition of tRNA or glycogen during this passage, it is not obligatory and the addition can be also performed during the previous passages but before the precipitation with isopropanol. Glycogen concentrations must be at least 10 ng/ml, preferably between 10 and 200 ng/ml and better between 50 and 100 ng/ml. The preferred application is the addition of glycogen both in this passage and in passage a), immediately before or contemporary to the addition of the lysis solution to the sample
d) DNA is isolated from the organic phases stored during the previous passages. Precipitation is carried out using a short chain aliphatic alcohol, preferably ethanol and by a precipitating agent such as glycogen (precipitation occurs after a short incubation at room temperature). The residual phenol can be removed by washing in saline solution (preferably NaCl or Na-citrate) at a concentration of 10-200 mM, preferably 80-120 mMa nd better 100 MM, containing at least 5% of a short chain aliphatic alcohol, preferably ethanol. Washing should be repeated at least two-three times.

The invention includes the realization of an extraction kit for the simultaneous isolation and separation of RNA and DNA from fresh, frozen, autoptical or paraffin-embedded biological samples, according to the new method. The kit consists a tube with lysis solution, a tube with the precipitating agent, a tube with ribonucleases inhibitor etc, and instructions describing the method in detail. Optionally sterile tubes treated with ribonuclease inhibitors, disposable blades etc. can be included in the kit.

Alternatively, the invention includes a kit for the extraction of viral nucleic acids from fresh, frozen, autoptical or paraffin-embedded biological samples, according to the new method. The kit includes some tubes with specific oligonucleotides for the revelation of viral agents by PCR, instructions describing the new method in detail and optionally some tubes containing reagents for reverse transcriptase (i.e. RNA-dependent DNA-polymerase, random primers, oligo(dT)esanucleotides).

### BRIEF DESCRIPTION OF THE DRAWINGS

FIG. 1. Glyceraldehyde-3-phosphate dehydrogenase (G3PDH) amplification by RT-PCR of RNA extracted using the new method.

The figure shows electrophoresis on agarose gel of RNA fragments obtained by RT-PCR from samples treated with the new method. Proteinase K digestion lasts 72 hours. Lane 1: G3PDH RT-PCR of sample No. 27 (72 hours lysis ); Lane 2: G3PDH RT-PCR of sample No. 27 (12 hours lysis ); Lane 3: G3PDH RT-PCR with reagents, without RNA (negative control); Lane 4: DNA marker (Factor VIII).

FIG. 2. β-globin amplification by PCR of DNA extracted using the new method.

The figure shows electrophoresis on agarose gel of DNA fragments obtained by RT-PCR from samples treated with the new method. Proteinase K digestion lasts 72 hours. Lane 1: β-globin PCR of sample No. 27 (72 hours lysis ); Lane 2: β-globin PCR of sample No. 27 (12 hours lysis ); Lane 3: β-globin PCR with reagents, without DNA (negative control); Lane 4: DNA marker (Factor VIII).

FIG. 3. Viral amplification by PCR of DNA and RNA extracted with the new method.

The figure shows electrophoresis on agarose gel of DNA and RNA extracted and amplified respectively, for Enterovirus and Adenovirus. Lane 1: DNA marker; Lane 2: G3PDH RT-PCR of sample No. 1 (RNA extraction control); Lane 3: RT-PCR for Enterovirus sample No. 1; Lane 4: G3PDH RT-PCR of sample No. 83 (RNA extraction control); Lane 5: RT-PCR for Enterovirus sample No. 83; Lane 6: G3PDH RT-PCR of sample No. 3 (RNA extraction control); Lane 7: RT-PCR for Enterovirus sample No. 3; Lane 8: β-globin PCR of sample No. 86 (DNA extraction control); Lane 9: PCR for Adenovirus sample No. 86; Lane 10: RT-PCR for Enterovirus: KB cell infected with Coxsackievirus B3 (positive control); Lane 11: RT-PCR for Enterovirus with reagents, without RNA (negative control); Lane 12: PCR for Adenovirus with cells infected with Adenovirus (positive control); Lane 13: PCR for Adenovirus with reagents and without DNA (negative control).

FIG. 4. PCR Viral amplification by PCR of DNA and RNA extracted with the new method.

Electrophoresis on agarose gel of DNA and RNA extracted and amplied respectively for Enterovirus and Adenovirus on autoptic tissue. Lane 1: DNA marker; Lane 2: G3PDH RT-PCR (234bp) sample No. 108 (RNA extraction control); Lane 3: RT-PCR for Enterovirus (392 bp) sample No. 108; Lane 4: β-globin PCR (269 bp) sample No. 106 (DNA extraction control); Lane 5: PCR for Adenovirus (308 bp) samples No. 106 and No. 114; Lane 6: RT-PCR for Enterovirus: KB cell infected with Coxsackievirus B3 (positive control); Lane 7: RT-PCR for Enterovirus with reagents, without RNA (negative control); Lane 8: PCR for Adenovirus with cells infected with Adenovirus (positive control); Lane 9: PCR for Adenovirus with reagents and without DNA (negative control).

FIG. 5. PCR Viral amplification by PCR of DNA and RNA extracted with the new method.

Detection of RNA virus (HCV) in a heart fragment formalin-fixed for one year. Lane 1: DNA marker (Factor VIII); Lane 2: Sample No. 40; Lane 3: Sample No. 41; Lane 4: Sample No. 42; Lane 5: Sample No. 43; Lane 6: Sample No. 44; Lane 7: Extraction negative control; Lane 8: Positive control for HCV.

### EXPERIMENTAL SECTION

Reagents:

Xylene (CARLO ERBA)
Absolute ethanol (MERCK)
Deionized steril water
DEP water (water treated with 0.1% diethyil pyrocarbonate for at least 12 hours gently agitated followed by heating at 100° C. for 1 min or after autoclave processing)
Guanidine isothiocyanate (SIGMA)
2-Mercaptoethanol (SIGMA)
Lauril-sarcosyne (SIGMA)
Proteinase K (DNAse-RNAse free) (BOEHRINGER)
Vanadil Ribonucleaside complex (SIGMA)
Glycogen (BOEHRNGER)
Phenol: Chloroform 5:1 pH 4.7 (SIGMA)
Chloroform (SIGMA)
Isopropanol (MERCK)
0.5 M NA Citrate pH 7 (BAKER)
75% Ethanol (MERCK)
   Material
Microtome
Microtome Knives
RNAse-DNAse free tube (EPPENDORF)
RNAse-DNAse free tips (EPPENDORF)
Pipette (EPPENDORF)
Spectrophotometer (λ5 PERKIN ELMER)
Thermal cycler (2400 PERKIN ELMER)
   Samples

Both liquid nitrogen frozen and 10% buffered formalin and/or paraffin embedded fragments biopsies from different tissues were used. In particular the following samples were processed:
a) 25 liquid nitrogen frozen biopsies (1-9 mg) (10 endomyocardial biopsies, 5 liver biopsies, 5 skin biopsies and 5 transbronchial biopsies),
b) 25 liquid nitrogen frozen fragments (10-20 mg) (10 myocardial fragments, 10 lung fragments, 5 thyroid fragments)
c) 25 formalin fixed (time of fixation: <12 h) and paraffin embedded biopsies (tissue area:3-20 mm²) (10 endomyocardial biopsies, 5 transbronchial biopsies, 5 skin biopsies, 5 gastric biopsies)
d) 30 formalin fixed (time of fixation: from 3 to maximum 7 days) and paraffin embedded tissue fragments (tissue area: 30-80 mm²) (10 liver fragments, 8 gastric fragments, 7 skin fragments and 5 myocardial fragments)
e) 15 autoptic formalin-fixed (time of fixation: from 3 to maximum 7 days) and paraffin embedded tissue fragments (5 myocardial fragments, 5 lung fragments and 5 liver fragments)

For frozen tissue fragments weighing more than 1 mg a gentle fragmentation with lancet was performed and rapidly transferred into the lysing solution. 15-20 sections of 10μ for the biopsies and one to four sections of the same thickness for the fragments were used. Formalin-fixed paraffin-embedded biopsies and fragments were randomly chosen. All the samples were always processed by the same lab workers.

### EXAMPLE 1

### RNA Extraction from Frozen and/or Fixed Fragments or Biopsies

Frozen biopsies (1 to 9 mg) in particular frozen biopsies of 1.5 mg and fixed paraffin-embedded biopsies (3 to 20 mm²) in particular mean of 10 mm² were diced and transferred into 400 μl lysing solution (2 M Guanidine isothiocyanate, 0.1 mM 2-Mercaptoethanol, 25 mM Na citrate pH 7, 0.5% N-Lauril-sarcosine and proteinase K (2 mg/ml for frozen biopsies and 5 mg/ml for fixed biopsies). Vanadyl ribonucleaside complex (4 μl) and glycogen (1 μl) were added and the solution was incubated at 40° C. overnight.

Formalin-fixed fragments of about 60 mg, before dried in a desiccator oven (for 2 h at 30-35° C.) and then treated with alumina (the same weight of the dried sample). After few min, the mixed alumina tissue was incubated in lysing solution and processed following the method. The efficacy of the method was well documented by the detection of HCV in a formalin fixed myocardial fragment (formalin-fixation for about 1 year) (FIG. 5).

Formalin-fixed and paraffin-embedded sections of 10 μm of thickness (15-30 sections for biopsies and 1-4 sections for fragments) were transferred in a DNAse RNAse free tube and 1 ml of xylene at 37° C. for 20 min were used for deparaffinisation. After centrifuging at 12000 rpm at 4° C. for 3 min the supernatant was removed, fresh xylene added and steps repeated. Two identical washes were performed with 1 ml of absolute ethanol for 5 min at room temperature, centrifuging at 12000 at 4° C. for 3 min, followed by air drying of the tissue pellet.

The pellet was resuspended in 400 μl lysing solution and incubated at 37° C. overnight. If the tissue was not completely digested another 80-100 μg of proteinase K was added and the tissue was incubated at 37° C. for 24 hours.

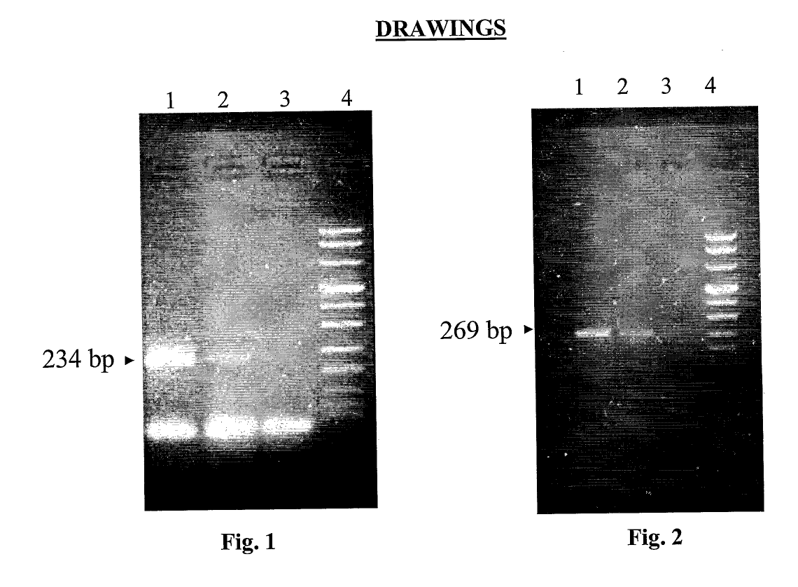

For archival tissues represented by fragments (from 30 mm² to more than 1 cm² ) the digestion was prolonged up to 72 hours thus obtaining a more successful amplification for G3PDH (housekeeping gene) (FIG. 1a).

Both frozen and archival tissues were then processed following the same protocol:

The same volume of phenol/chloroform (5:1, pH: 4.7) was added, mixed via repeated inversion and then centrifuged at 12000 rpm at 4° C. for 5 min. The supernatant (aqueous phase containing RNA) was transferred into a new tube. After adding 100-200 μl of DEPC water to the original tube, the sample was mixed via inversion and centrifuged at 12000 rpm at 4° C. for 5 min then the supernatant was transferred to the same original tube and the organic phase was stored at 4° C. for the DNA extraction. Subsequently the same volume of phenol/chloroform was added, mixed via immersion and then centrifuged at 12000 rpm for 5 min. The aqueous phase was transferred into a new tube and the organic phase stored at 4° C. for the next DNA extraction. Then the same volume of chloroform was added to the aqueous phase, gently mixed by inversion and centrifuged at 12000 rpm for 5 min. The aqueous phase was then transferred into a new tube and the same volume of isopropanol was added. After repeated inversion it was placed at −80° C. for 1 hour. The precipitated RNA was pelleted by centrifuging at 12000 rpm at 4° C. for 15 min, the isoproponanol was discarded and the pellet was washed with cold eythanol (75%), then air dried and finally resuspended in 20 μl DEPC water. The nucleic acid was measured by a spectrophotometer and stored at −80° C. RNA has been extracted both from the frozen and fixed paraffin-embedded samples. The RNA quantity was measured by a spectrophotometer obtaining the following ranges: from 0.76 μg/μl to 1.57 μg/μl for frozen biopsies and from 0.12 μg/μl to 0.99 μg/μl for fixed paraffin-embedded biopsies (Tables 5 and 6) in 20 μl of total solution. A mean from 15.2 to 31.4 μg of RNA was extracted from 1-9 mg of frozen tissue by using this new method. The best results has been obtained from frozen liver samples (from 0.93 to 1.57 μg/μl ). The weight of the frozen biopsies did not significantly influence the obtained RNA quantity; indeed more RNA was obtained using smaller biopsies of the same type tissue. The ratio A260/280 showed good values (from 1.5 to 2.0) in all frozen and fixed samples (Tables 5 and 6).

RNA was successfully extracted from all frozen and fixed fragments. RNA ranged from 1.22 μg/μl to 2.69 μg/μl for frozen tissues and from 0.44 μg/μl to 1.33 μg/μl for fixed tissues. In summary from 24.4 to 53.8 μg of RNA was extracted from 10-20 mg of tissue. The ratio A260/280 showed good values (from 1.5 to 2.0) in all frozen and fixed fragments.

### EXAMPLE 2

### DNA Extraction from the Organic Phase Obtained Following the Example 1

The tubes containing the organic phase stored at 4° C. (as obtained following RNA extraction as described in the example 1) were processed for DNA extraction. The aqueous phase was completely removed and the DNA precipitated from the organic phase by adding 1 μl of glycogen, 200 μl of absolute ethanol mixed by inversion for 2-3 min at room temperature and then centrifuged at 12000 rpm at 4° C. for 5 min, settling the supernatant.

The eventual presence of phenol was removed by adding 0.1 M of citrate Na in 10% ethanol (100 μl in 100 of lysing solution). After incubation for 30 min at room temperature the sample was centrifuged at 12000 rpm at 4° C. for 5 min and the supernatant then settled.

Washing with citrate Na was repeated twice and finally the pellet was washed with 200 μl of 75% ethanol (for 100 μl of lysing solution). The two pellets obtained were dried and resuspended in sterile water and unified in the same tube. The extracted DNA was stored at −4° C. until use.

The DNA was extracted in all both frozen and formalin-fixed biopsies in the following range: from 0.09 μg/μl to 0.2 μg/μl for frozen biopsies (Table 5) and from 0.01 to 0.08 for fixed biopsies (Table 6). This new method was able to extract 1.8-3.8 μg of DNA from 1-9 mg of frozen tissue. No difference in terms of DNA quantity was observed among different types of tissues. The weight of the biopsy did not influence the quantity of extracted DNA. The ratio A260/280 gave good results (ranging from 1.5 to 1.8) in 16/25 frozen biopsies (64%) and fixed samples (80%).

The DNA was extracted from all frozen and fixed fragments and ranged from 0.29 μg/μl to 0.67 μg/μl for frozen tissues and from 0.08 to 0.41 for fixed tissues. Our protocol was able to extract from 5.8 to 13.4 μg of DNA from 10-20 mg of tissue. The quantity of DNA extracted using the Blin and Stafford method and Omnizol kit was less (0.1 to 0.19 μg/μl and 0.1 to 0.32, respectively—Table 5—).

### EXAMPLE 3

### PCR of Nucleic Acids Extracted Using the New Method

For evaluation of the quality of the extracted nucleic acids, PCR for house-keeping genes was performed; β-globin and glyceraldehydes-3-phosphate dehydrogenase (3GPDH) for DNA and RNA, respectively. The primers (maximum 21 base pairs) used for PCR were purified in HPLC (Amersham Pharmacia Biotech).

PCR and retro-transcription specifics are reported in Tables 2,3 and 4. The PCR products were visualised on an NU-SIEVE 3:1 gel and UV photographed.

Successful amplification for β-globin was obtained in all frozen and fixed samples. Enteroviral and adenoviral genomes were also investigated in nucleic acid extracted from diagnostic samples (15 samples: 10 biopsies and 5 autoptic fragments).

All the specifics of the primers including the number of base pair and annealing temperature are reported in Table 1.

TABLE 1
Nucleotides used for PCR

| | | Annealing |
|---|---|---|
| Type | Amplicon | Temperature |
| G3PDH^{#} | 234 bp | 50° C. |
| βglobina* | 269 bp | 44° C. |
| Enterovirus^{⋄} | 391 bp | 55° C. |
| Adenovirus° | 308 bp | 57° C. |

^{#}reference: Ercolani L et al. J. Biol. Chem. 1988; 263: 1535-41.
*reference: Saiki RK et al. Science 1985; 230: 13450-4
^{⋄}reference: Gamma RE et al. J Med. Virol. 1989; 28: 73-7.
°reference: Lozinski GM et al. Hum. Pathol. 1994; 25: 831-834.

Specifics about PCR are reported in Table 2.

TABLE 2
PCR Amplification Reaction

| Reagents | Concentration | Quantity | |
|---|---|---|---|
| MgCl₂ 25 mM | 2.5 mM | 5 | μl |
| Buffer* 10X | 1X | 5 | μl |
| “Primers” 20 pmoli/μl | 20 pMol (each) | 1 | μl |
| Taq polymerase 5 U/μl | 1.2 U | 0.25 | μl |
| dNTP 10 mM | 200 μM (each) | 1 | μl (each) |

*Reaction buffer used for Taq polymerase (Perkin Elmer)

In a total final volume of 50 μl (the volume was reached by adding deionized sterile water) 1 μg of DNA was added.

The following steps were used for each amplification:

| | | | |
|---|---|---|---|
| Initial denaturation: | 3′ | 94° C. | 1X |
| Denaturation : Annealing : Extension : | 1′1′1′ | 94° C. (T° specific) 72° C. | 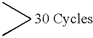 |
| Final extension : | 7′ | 72° C. | 1X |

The RNA extracted as described in example 1 was retro transcribed and then amplified for G3PDH. The steps for retro transcription are reported in Table 3.

TABLE 3
Retrotranscription

| Reagents | Concentration | Quantity |
|---|---|---|
| MgCl2 | 5 mM | 4 μl |
| Buffer | 1X | 2 μl |
| “Primer downstream” | 20 pMol | 1 μl |
| RNAsin 20 U/μl | 1 U/μl | 1 μl |
| Deossinucleotidi | 1 mM (each) | 2 μl (each) |
| MuLV Reverse | 2.5 U/μl | 1 μl |
| Transcriptase 50 U/μl | | |

*Reaction buffer: buffer used for MuLV enzyme (Perkin Elmer).

For each retrotranscription (final volume 20 μl) at least 1 μg of RNA was used.

The following steps were followed:
Retro transcription 50′ 42° C.
Enzyme denaturation 5′ 99° C.

The sample was then stored at 4° C. until PCR.

All the specifics regarding PCR of cDNA are summarized in Table 4.

TABLE 4
Amplification of cDNA

| Reagents | Concentration | Quantity |
|---|---|---|
| MgCl2 | 2 mM | 2 μl |
| Buffer* | 1X | 4 μl |
| “Primer upstream” | 20 pMol | 1 μl |
| Taq polymerase | 1.2 U | 0.25 μl |

20 μl of cDNA was used in a 100 μl PCR reaction using the following steps:
Initial denaturation 2′ 95° C.
Denaturation 30 sec 95° C.
Annealing 30 see (T° spec)
Extension 1′ 72° C.
Final extension 7′ 72° C.

The PCR product was then stored at 4° C.

RNA: successful amplification for G3PDH was obtained from all RNAs extracted from frozen fragments and in 42/45 (93%) fixed fragments: no amplification was obtained in 3 autoptic formalin-fixed tissues.

Positive controls for G3PDH used in the same reactions confirmed that negative results were true negative (no efficient RNA extraction).

RT-PCR was repeated at least 3 times in negative cases: 1) with the same quantity of RNA used in the previous reaction, 2) doubling the RNA, 3) halving the RNA. No variation was obtained.

DNA: successful amplification for β-globin in all frozen fragments and in 41/45 (91%) fixed fragments. Positive controls for β-globin used in the same reactions confirmed that negative results were true negative (no efficient DNA extraction).

PCR was repeated at least 3 times in negative cases: 1) with the same quantity of DNA used in the previous reaction, 2) doubling the DNA, 3) halving the DNA. No variation was obtained.

For archival tissue represented by fragments (from 30 mm² to >1 cm²) the digestion with lysing solution was prolonged for a time between 24 to 72 hours obtaining better β-globin amplification (FIG. 2).

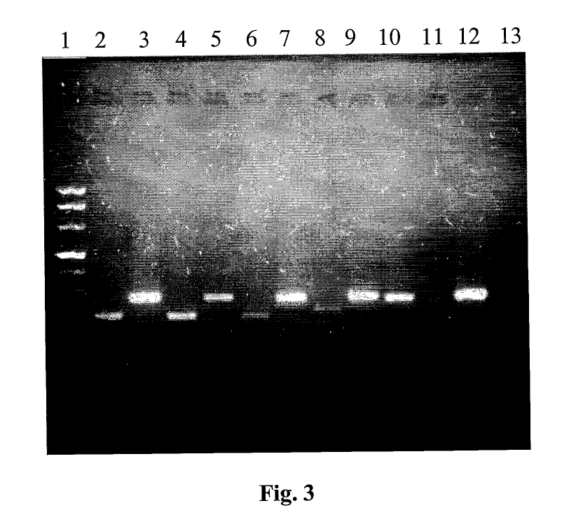

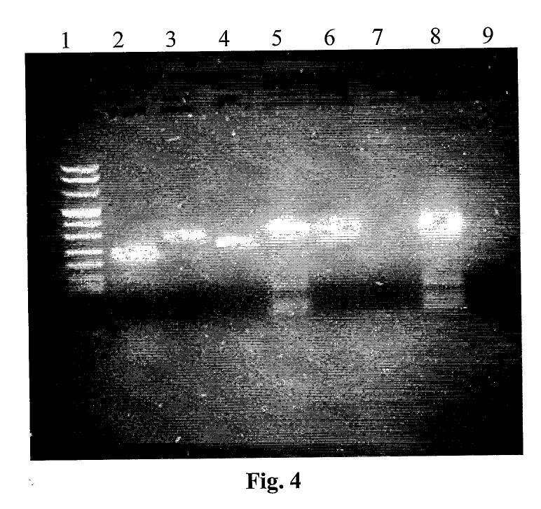

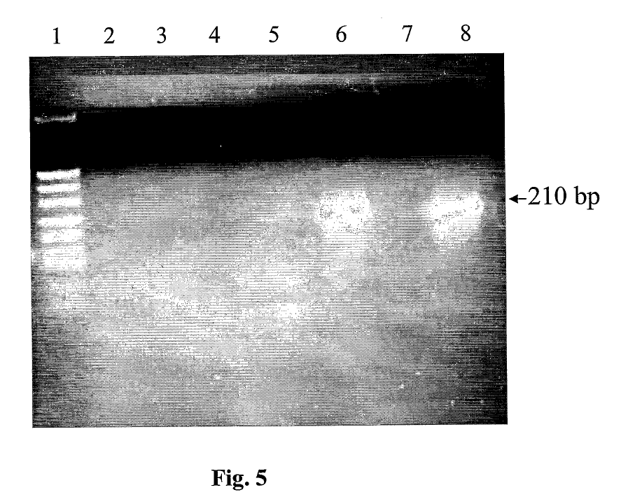

Enteroviral and adenoviral genomes were detected in 4 endomyocardial samples (2 frozen biopsies and 2 formalin-fixed biopsies) (FIG. 3). Successful viral amplification was also obtained in 3 autoptic myocardial and lung fragments from patients affected by myocarditis and pneumonia, respectively (FIG. 4).

### EXAMPLE 4

### Comparison Between the Quantity of Nucleic Acid Extracted Following the New Method and that Obtained Using Well Known Methods

In each sample the nucleic acids were analyzed both by spectrophotometry and by PCR for house-keeping genes (see previous example).

In particular DNA was extracted from frozen tissue following the method (lysing solution: EDTA,TRIS-HCL and proteinase K) reported by Sambrock and Maniatis, CSH 1988 (Blin and Stafford, Nucleic Acids Res., 1973; 3:2303). RNA was extracted from frozen tissue using Chomczynski and Sacchi (Chomczynski P and Sacchi N., Anal. Biochem; 1987; 162:156-159): the name of the commercial kit is RNAzol. The Omnizol kit (able to extract both nucleic acids) was also used in frozen tissue.

In the following tables the value of nucleic acids obtained with the new method in comparison with the other protocols are reported (memo: the weight of fragments was from 10 to 20 mg and for biopsy from 1 to 9 mg). In all cases all the concentration of nucleic acids for a total 20 μl volume are also reported.

TABLE 5
Quantity of nucleic acid: comparison of the new method,
RNAzol, Omnizol and Blin & Stafford

| Nucleic | | | Fragment |
|---|---|---|---|
| Acid | Protocol | Biopsy (1-9 mg) | (10-20 mg) |
| RNA | New method | 15.2-31.4 μg | 24.4-53.8 μg |
| RNA | OMNIZOL | 1.2-5.2 μg | 4.4-11.6 μg |
| RNA | RNAZOL | 0.2-3.0 μg | 2.0-6.6 μg |
| DNA | New method | 1.8-3.8 μg | 5.8-13.4 μg |
| DNA | OMNIZOL | 0.8-2.2 μg | 0.1-0.32 μg |
| DNA | Blin & Stafford Method | 0.2-0.8 μg | 0.1-0.19 μg |

From 0.76 μg/μl to 1.57 μg/μl for frozen biopsies (Table 5) and from 0.12 μg/μl to 0.99 μg/μl for fixed biopsies in 20 μg/μl of total solution were extracted using the new method and are reported in Table 6. As reported in Table 5 the quantity of RNA obtained from frozen biopsies using RNAzol and Ominzol was much lower than the values obtained using the new method (from 0.01 to 0.15 μg/μl -0.2-3 μg in total—using RNAzol and from 0.06 to 0.26 μg/μl −1.2 to 5.2 μg in total—using Omnizol ). Also the values obtained from frozen fragments were dig ran lunga less when Simultaneous was compared with the other methods (from 0.1 to 0.33 μg/μl -2-6.6 μg in total for RNAzol and from 0.22 to 0.58 μg/μl (4.4 to 11.6 μg in total).

DNA was successfully extracted from all frozen and fixed biopsies using the new method ranging from 0.09 μg/μl to 0.2 μg/μl for frozen biopsies (Table 5) and from 0.01 to 0.08 μg/μl for fixed biopsies (Table 6). The values of DNA extracted using the other protocols were much lower: from 0.04 μg/μl to 0.11 μg/μl (0.8 to 2.2 in total) using the Blin and Stafford method and from 0.01 to 0.04 μg/μl (0.2-0.8 in total) using Omnizol (Table 5).

The values of RNA and DNA obtained from fixed samples are reported in Table 6. The extraction was performed only using this new method because RNAzol and Omnizol are not adoptable for extraction from fixed tissue samples.

TABLE 6
Nucleic acid values extracted from fixed samples using the new method

| Nucleic acid | Biopsy | Fragments |
|---|---|---|
| RNA | μg 2.4-19.8 | μg 8.8-26 |
| DNA | μg 0.2-1.6 | μg 1.6-8.2 |

RNA and DNA extracted using this new method from different types of tissues (myocardium, liver, skin, lung, stomach and thyroid, both frozen and fixed) were also compared.

Moreover autoptic formalin-fixed paraffin-embedded tissues were also analyzed.

TABLE 7
Nucleic extraction values from different types of tissues

| Type of | | Nucleic | Concentration | |
|---|---|---|---|---|
| tissue | Specimen | Acid | (μg/μl) | A260/280 |
| Myocardium | Biopsies (frozen) | RNA | 1.054 | 1.86 |
| | | DNA | 0.118 | 1.48 |
| | Biopsies (fixed) | RNA | 0.428 | 1.83 |
| | | DNA | 0.032 | 1.54 |
| | Fragments (frozen) | RNA | 1.795 | 1.91 |
| | | DNA | 0.41 | 1.51 |
| | Fragments (fixed) | RNA | 1.145 | 1.95 |
| | | DNA | 0.13 | 1.45 |
| | Autoptic fragments | RNA | 0.326 | 1.66 |
| | | DNA | 0.052 | 1.46 |
| Liver | Biopsies (frozen) | RNA | 1.13 | 1.88 |
| | | DNA | 0.176 | 1.5 |
| | Fragments (fixed) | RNA | 0.595 | 1.62 |
| | | DNA | 0.234 | 1.48 |
| | Autoptic fragments | RNA | 0.516 | 1.82 |
| | | DNA | 0.138 | 1.5 |
| Skin | Biopsies (frozen) | RNA | 0.972 | 1.88 |
| | | DNA | 0.114 | 1.48 |
| | Biopsies (fixed) | RNA | 0.83 | 1.84 |
| | | DNA | 0.042 | 1.52 |
| | Fragments (fixed) | RNA | 0.92 | 1.81 |
| | | DNA | 0.128 | 1.5 |
| Lung | Biopsies (frozen) | RNA | 0.992 | 1.84 |
| | | DNA | 0.13 | 1.48 |
| | Biopsies (fixed) | RNA | 0.65 | 1.84 |
| | | DNA | 0.042 | 1.58 |
| | Fragments (frozen) | RNA | 1.791 | 1.84 |
| | | DNA | 0.446 | 1.55 |
| | Autoptic fragments | RNA | 0.472 | 1.84 |
| | | DNA | 0.076 | 1.46 |
| Stomach | Biopsies (fixed) | RNA | 0.622 | 1.84 |
| | | DNA | 0.03 | 1.56 |
| | Fragments (fixed) | RNA | 0.815 | 1.81 |
| | | DNA | 0.135 | 1.51 |
| Thyroid | Fragments (frozen) | RNA | 1.896 | 1.84 |
| | | DNA | 0.404 | 1.54 |

1. A method of simultaneously isolating both nucleic acids (RNA and DNA) from the same sample weighing not less than 0.5 mg—fresh, frozen, fixed or autoptic—including the following steps:
a) digestion of a sample incubated in a lysing solution consisting of: a caotropic agent, a ionic detergent, a proteolytic enzyme and a reducing agent;
b) enzyme inactivation by using extraction with a mixture of aromatic alcohols and producing an organic phase, that was stored and added to a second organic phase;
c) precipitation of RNA by adding a precipitating agent to the aqueous phase and an aliphatic short chain alcohol;
d) precipitation of DNA from the organic phase as reported in step b) by using a precipitating agent and a short chain aliphatic alcohol.

2. Method in agreement with claim 1 where the lysing solution used in step a) includes:
a caotropic agent, either urea or guanidine thyocianate;
a ionic detergent, either SDS or SLS;
a proteolytic enzyme: proteinase K, trypsin, chymotrypin, pepsin or pronase;
a reducing agent, either β-mercaptoethanol or ditiotreitol.

3. Method in agreement with claim 1 including also the addition of RNAse inhibitor in step a), b), or c) of the method, alternatively.

4. Method in agreement with claim 3 where the inhibitor is a Vanadyl ribonucleoside complex.

5. Method in agreement with claims 1-4 including a nucleic acid precipitating agent, either tRNA or glycogen: alternatively added in step c) or step a) and also in step d) of the method

6. Method in agreement with claim 5 wherein the precipitating agent is glycogen.

7. Method in agreement with claim 6 where the final concentration of glycogen is no less than 10 ng/ml.

8. Method in agreement with claim 7 wherein the final concentration of glycogen is no less than 50 ng/ml.

9. Method in agreement with claim 1 wherein the short chain aliphatic alcohol is isopropanol or ethanol.

10. Method in agreement with claim 2 wherein the guanidine salt in lysing solution of step a) is selected from the group consisting of guanidine thiocyanate and guanidine hydrochloride using a concentration ranging from 1 to 4 M.

11. Method in agreement with claim 2 wherein the proteolytic enzyme in the lysing solution of step a) is proteinase K.

12. Method in agreement with claim 11 wherein the concentration of proteinase K ranges from 0.1 to 10 mg/ml and wherein the incubation with this enzyme is performed at a temperature more than 20° C.

13. Method in agreement with claim 1 including at the end of step a) of the method a supplementary addition of the proteolytic enzyme with subsequent incubation.

14. Method in agreement with claim 1 wherein the miscela of organic solvent and aromatic alcohol of step b) is phenol or a phenol/chloroform solution at acid pH, mainly between 5 and 6, and more preferably 5.5.

15. Method in agreement with claim 14 wherein the volume ratio of phenol and chloroform is from 3:1 to 7:1 in the solution.

16. Method in agreement with claim 1 wherein the aqueous phase is re-extracted with chlorophorm after the first extraction with aromatic alcohol following step b) of the method.

17. Method in agreement with claim 1 wherein the excess salt is removed from the RNA precipitate obtained at step c) washing the pellet with a short chain alcohol diluted with deionised water.

18. Method in agreement with claim 1 where the aqueous solution at step b) and the deionised water are treated with DEPC.

19. Method in agreement with claim 1 where the aliphatic alcohol added to precipitate DNA in agreement with step d) of the method is isopropanol and the precipitation is performed incubating the sample at a temperature lower than 0° C.

20. Method in agreement with claim 19 wherein precipitated DNA is washed with a saline solution including at least 5% of organic solvent and wherein this step is optionally repeated to remove traces of phenol from precipitated DNA.

21. Method in agreement with claim 20 wherein the saline solution is either citrate or Na Cl.

22. Method in agreement with claim 21 wherein the solution is Na citrate at a concentration between 10 and 200 mM with pH from 6.8 to 7.3.

23. Method in agreement with claim 1 wherein the biological material is represented by cell culture, tissue biopsy, tissue fragment or optionally by paraffin-embedded sections.

24. Method in agreement with claim 23 wherein the paraffin-embedded sections are first deparaffinised using an organic solvent

25. Method in agreement with claim 24 wherein the organic solvent is either xylene or benzene-derived.

26. Method in agreement with claim 23 for extraction of viral nucleic acids from biological materials.

27. Method in agreement with claim 23 wherein the nucleic acid is RNA.

28. Method in agreement with claim 1 wherein the sample weighs no more than 20 mg and wherein the volume of lysing solution, in agreement with step a) is from 100 to 800 μl.

29. Kit for simultaneous and separate extraction of RNA and DNA from fresh and fixed samples, optionally also paraffin-embedded, in agreement with the method following claim 1, including a tube with a lysing solution, a tube with a precipitating agent, a tube with a RNAse inhibitor and instructions describing the method in agreement with claim 1 and optionally sterile and RNAse free tubes, disposable knives and alumina.

30. Kit for extraction of viral nucleic acids from fresh, fixed or autoptic biological samples, optionally also paraffin-embedded, in agreement with the method following claim 1 including one or more tubes with oligonucleotides specific for viral identification using PCR, instructions describing the method in agreement with claim 1 and optionally tubes with reagents for retro transcription of RNA.
